(19) United States
(12) Patent Application Publication
(10) Pub. No.: US 2024/0382153 A1
(43) Pub. Date: Nov. 21, 2024

(54) OPIOID SENSORS

(71) Applicants: Oakland University, Rochester, MI (US); Auburn University, Auburn, AL (US); George Mason University, Fairfax, VA (US)

(72) Inventors: Zhe Wang, Rochester, MI (US); Xinyu Zhang, Auburn, AL (US); Pei Dong, Fairfax, VA (US)

(21) Appl. No.: 18/662,986

(22) Filed: May 13, 2024

Related U.S. Application Data
(60) Provisional application No. 63/466,565, filed on May 15, 2023.

(51) Int. Cl. A61B 5/00; A61B 5/1473; G01N 27/02; G01N 27/327

(52) U.S. Cl. CPC A61B 5/4845; A61B 5/1473; A61B 5/6833; A61B 5/6849; G01N 27/02; G01N 27/3276; A61B 2562/125

(57) ABSTRACT

An opioid sensor includes a film having two opposed surfaces. The film includes a conductive polymer, and an anchor molecule attached to the conductive polymer so that it is immobilized at a first of the two opposed surfaces. The anchor molecule is to adsorb an opioid. The opioid sensor also includes a working electrode at least partially in contact with a second of the two opposed surfaces; and a counter electrode or counter/reference electrode electrically connected to, and positioned a spaced distance from, the working electrode.

### CROSS-REFERENCE TO RELATED APPLICATIONS

This application claims the benefit of U.S. Provisional Application Ser. No. 63/466,565, filed May 15, 2023, the content of which is incorporated by reference herein in its entirety.

### BACKGROUND

Opioids are substances that act on opioid receptors on nerve cells to produce pain relieving effects and/or pleasure enhancing effects (e.g., euphoria). Thus, opioids are beneficial for treating moderate to severe pain. In contrast, however, opioids can bring on moderate risks and/or side effects, such as drowsiness, confusion, nausea, slowed breathing, and/or constipation, to more serious risks and/or side effects, such as addiction, overdose, and/or death. In recent years, opioid abuse has led to an overwhelming number of overdoses and deaths.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a semi-schematic and perspective view of an example of the opioid sensor disclosed herein attached to skin to adsorb human sweat;

FIG. 1B is a top semi-schematic view of another example of an opioid sensor that is to receive a liquid sample, such as human tears, human serum (e.g., blood, sweat, etc.), or a dissolved sample of a substance;

FIG. 1C is a cross-sectional view of the circled area in FIG. 1B;

FIG. 1D is a cross-sectional view of another example of an opioid sensor that is to receive a liquid sample or a dissolved sample of a substance;

FIG. 2 is a chemical equation depicting the attachment of arginine to polyaniline;

Each of FIG. 3A through FIG. 3K depicts a different chemical structure of an anchor molecule of a film to be used in a morphine sensor;

FIG. 4A through FIG. 4D each depict different examples of needle opioid sensors;

FIG. 5A is graph depicting the response, in terms of phase angle per the electrode surface area (“R” in degrees·cm², Y-axis) versus the log of frequency (Hz, X-axis), of a fentanyl sensor at various concentrations of fentanyl in a phosphate buffered saline (PBS) solution;

FIG. 5B is a graph depicting the fentanyl response from FIG. 5A at 10 Hz, in terms of a change in the phase angle per the electrode surface area (“ΔR” in degrees·cm², Y-axis) versus the fentanyl concentration (x 10³ nM, X-axis), illustrating that the response follows the Langmuir isotherm;

FIG. 5C is a graph depicting the linear Langmuir relationship (1/ΔR [x 10⁻³ deg⁻¹·cm²] (Y-axis) versus 1/C^{1-x} [x 10⁻³ nM⁻¹] (X-axis)) of the fentanyl sensor response in the PBS solution;

FIG. 5D is a graph depicting the linear Langmuir relationship (1/ΔR [x 10⁻³ deg⁻¹·cm²] (Y-axis) versus 1/C^{1-x} [x 10⁻³ nM⁻¹] (X-axis)) of the fentanyl sensor response in human blood, where the inset illustrates the data outlined in the box;

FIG. 5E is a graph depicting the linear Langmuir relationship (1/ΔR [x 10⁻³ deg⁻¹·cm²] (Y-axis) versus 1/C^{1-x [x} 10⁻³ nM⁻¹] (X-axis)) of the fentanyl sensor response in artificial tears;

FIG. 5F is a graph depicting the linear Langmuir relationship (1/ΔR [x 10⁻³ deg⁻¹·cm²] (Y-axis) versus 1/C^{1-x} [x 10⁻³ nM⁻¹] (X-axis)) of the fentanyl sensor response in artificial sweat;

FIG. 6A is a graph depicting the response, in terms of a change in the phase angle per the electrode surface area (“ΔR” in degrees·cm², Y-axis) versus the fentanyl concentration (x 10³ nM, X-axis) of a fentanyl sensor strip in human serum, where the inset depicts the linear Langmuir relationship (1/ΔR [x 10³ deg⁻¹·cm²] (Y-axis) versus 1/C^{1-x} [x 10⁻³ nM⁻¹] (X-axis)) of the sensor and a digital image depicting the flexibility of the sensor;

FIG. 6B is a graph depicting the response, in terms of a change in the phase angle per the electrode surface area (“ΔR” in degrees·cm², Y-axis) versus the fentanyl concentration (x 10³ nM, X-axis) of a fentanyl sensor strip exposed to artificial sweat on an artificial arm, where the inset depicts the linear Langmuir relationship (1/ΔR [x 10⁻³ deg⁻¹·cm²] (Y-axis) versus 1/C^{1-x} [x 10⁻³ nM⁻¹] (X-axis)) of the sensor and a digital image depicting the transparency of the sensor; and

FIG. 7 is a graph depicting the response, in terms of a change in the phase angle (Δφ, in degrees, Y-axis) versus the fentanyl concentration (M, X-axis) of a fentanyl needle sensor following Langmuir isotherm.

### DETAILED DESCRIPTION

The opioid sensors disclosed herein are based upon a film that is designed with a conductive polymer and a specific anchor molecule for the opioid that is to be sensed. The anchor molecule is capable of selectively adsorbing a particular opioid, and this selective and structural adsorption alters the electrochemistry in the conductive polymer. The structural interaction between the opioid and the anchor molecule is transduced to an electric property (e.g., charge) that resonates through the conductive polymer. Thus, the anchor molecule provides the film with opioid selectivity and specificity, and the conductive polymer effectively acts as a transducer and amplifier. The film is the sensing portion of each of the opioid sensors disclosed herein.

The opioid sensors can be used to detect a particular opioid in a liquid sample, such as human tears, human serum (e.g., blood, sweat, etc.), or in a dissolved sample of a substance (e.g., heroin suspected of containing fentanyl). It is to be understood that the term “sample” refers to both the liquid sample and the dissolved sample of a substance.

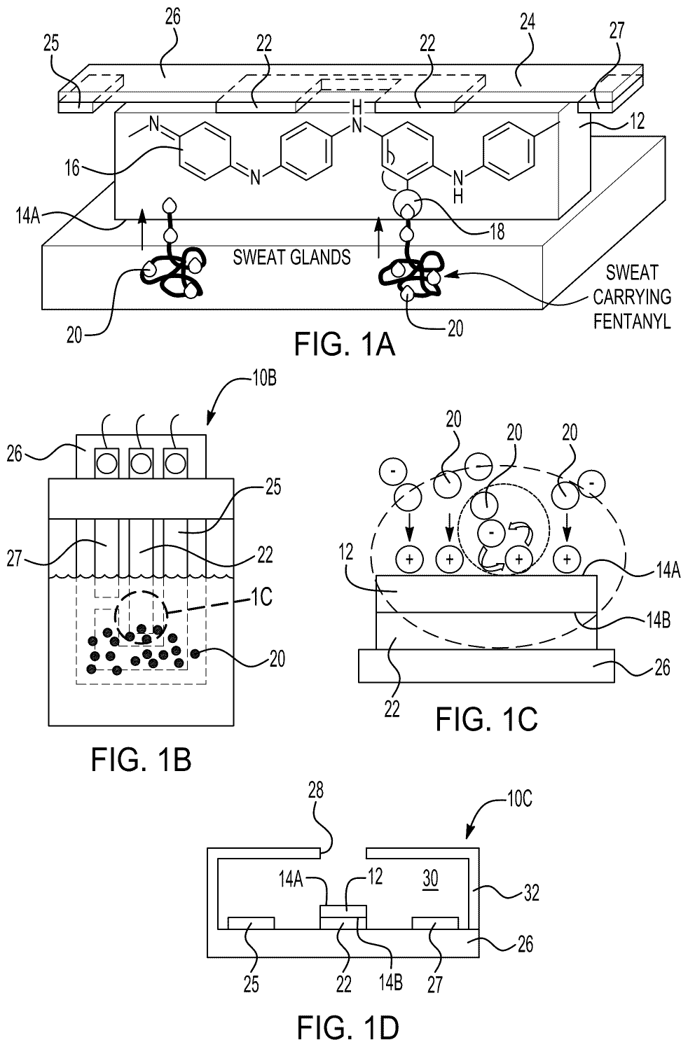

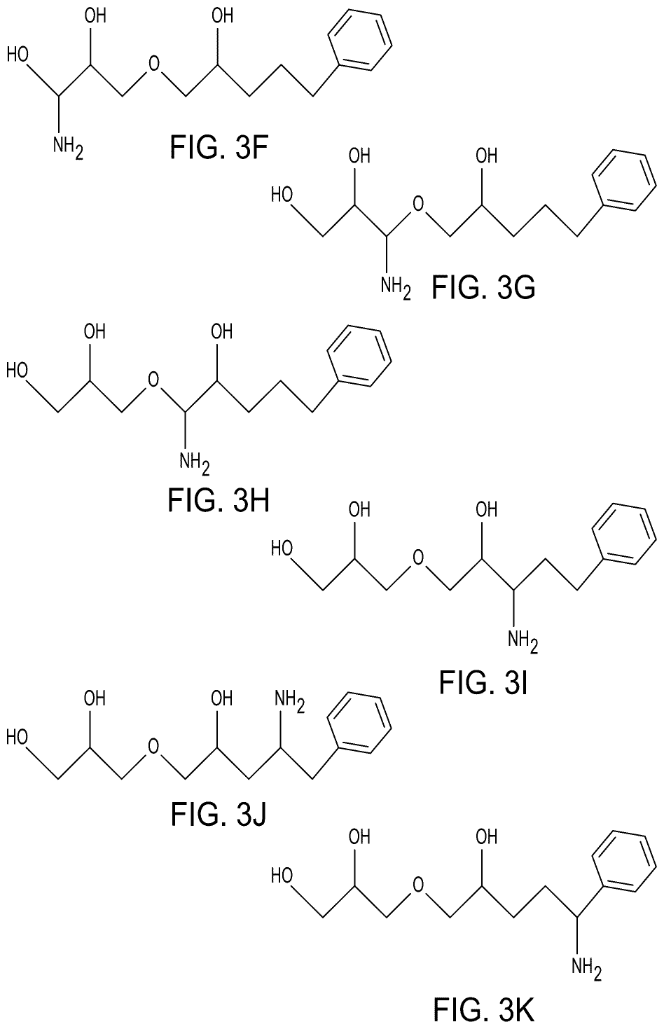

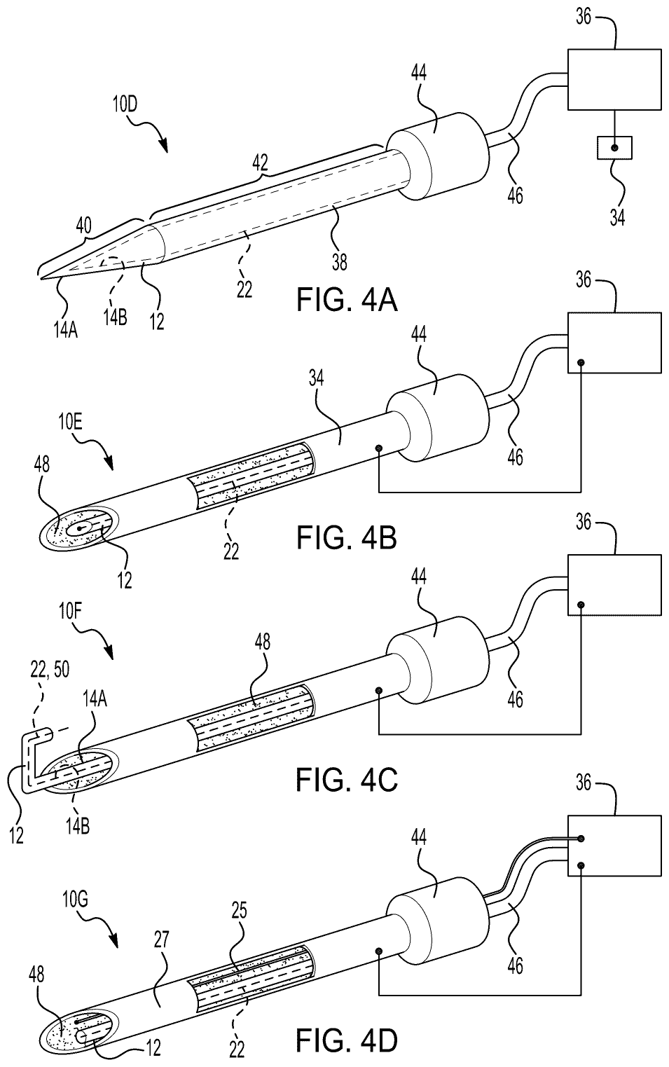

The film may be integrated into different types of opioid sensors including, for example, flexible portable devices, such as patches that adsorb fluid from a test subject (see FIG. 1A), test strips that adsorb fluid from a test sample (see FIG. 1B), or test strips or rigid devices that have a fluid insert port (see FIG. 1D). The film may also be integrated into a variety of needle opioid sensors 10D through 10G, examples of which are shown in FIG. 4A through FIG. 4D.

An example of the opioid sensor 10A that adsorbs fluid from a test subject is shown in FIG. 1A. The opioid sensor 10A may be in the form of a patch that can be temporarily secured to a test subject. This opioid sensor 10A may be used as a monitoring device when an opioid is used for pain management and treatment. As one example, a doctor could use the patch to monitor a patient's usage of the opioid. As another example, a patient could self-monitor the progress of his/her opioid treatment. The opioid sensor 10A may alternatively be may be in the form of a single use test strip. This opioid sensor 10A may be applied to a test subject to test for the presence of an opioid. As an example, an employer or law enforcement officer may use the opioid sensor 10A in a random drug test.

An example of the opioid sensor 10B that adsorbs fluid from a sample is shown in FIG. 1B. In this opioid sensor 10B, the sample is introduced to the exposed film 12 via submersion of at least a portion of the sensor 10B into the sample (as shown in FIG. 1B) or by dispensing the sample onto the exposed film 12 (e.g., using a manual or automated dispensing system). In the example of FIG. 1B, the surface of the opioid sensor 10B, including the film 12 and the electrodes 22, 25, 27, is exposed to the external environment. In another example, this opioid sensor 10B may be partially coated with a protective film, which is inert to the sample and to the sensing chemistry of the film 12. The protective film is configured with a sample input port (i.e., an opening) that is adjacent to, and thus exposes, the film 12.

Another example of the opioid sensor 10C that adsorbs fluid from a sample and that includes a sample input port 28 is shown in FIG. 1D. In this example, the film 12 is housed in a channel 30 formed between a substrate 26 and a lid 32 that is attached to the substrate 26. The sample input port 28 enables a sample to be introduced to the film 12 in the channel 30.

The opioid sensors 10B, 10C can be used for opioid point of care diagnosis in clinics and hospitals, for opioid identification and detection in the field of law enforcement, or for monitoring opioid treatment. These types of opioid sensors 10B, 10C may also be integrated into a medical device.

Other examples of the opioid sensor 10D, 10E, 10F, 10G are shown in FIG. 4A through FIG. 4D, respectively. These opioid sensors 10D, 10E, 10F, 10G are needle sensors that are used to test blood in vivo.

The sensors 10A, 10B, 10C, 10D, 10E, 10F, 10G may be collectively referred to as the sensors 10.

Each example of the opioid sensor 10 includes a film 12 having two opposed surfaces 14A, 14B, the film including: a conductive polymer 16 and an anchor molecule 18 attached to the conductive polymer 16 so that it is immobilized at a first of the two opposed surfaces 14A, the anchor molecule 18 to adsorb an opioid 20; and an electrode 22 positioned in contact with a second of the two opposed surfaces 14B.

The conductive polymer 16 may be any polymer that is electrically conductive and that is able to covalently attach to the anchor molecule 18. In an example, the conductive polymer 16 is selected from the group consisting of polyaniline, and a copolymer of polyaniline and polypyrrole. It is believed that other polyaniline derivatives or copolymers may be used as the conductive polymer 16. For example, the conductive polymer 16 may be poly (2,3-dimethylaniline), polyethoxyaniline, or poly (o-anisidine). Still other examples of the conductive polymer 16 include poly (3,4-ethylenedioxythiophene) (PEDOT), polyacetylene (PA), polypyrrole (PPy), polythiophene (PTH), poly (para-phenylene) (PPP), poly (phenylenevinylene) (PPV), and polyfuran (PF). Copolymers of any of the polymers set forth herein may be used as long as the copolymer is conductive and can attach the anchor molecule 18.

The anchor molecule 18 may be any molecule that is capable of adsorbing an opioid 20 and that is able to covalently attach to the conductive polymer 16. In an example, the anchor molecule 18 is selected from the group consisting of i)

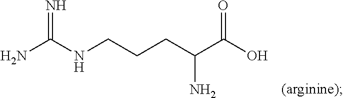 [SMILES: N=C(N)NCCCC(N)C(=O)O]

and ii)

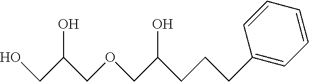 [SMILES: OCC(O)COCC(O)CCCc1ccccc1]

wherein —NH₂ is attached to a carbon of the phenyl ring or in place of one hydrogen of one —CH₂ in the linear chain. Specific examples of structure ii) are shown in FIG. 3A through FIG. 3K, which depict different and suitable positions for the amine group (—NH₂).

In one specific example, the opioid 20 to be sensed is fentanyl; the conductive polymer 16 is polyaniline; and the anchor molecule 18 is arginine:

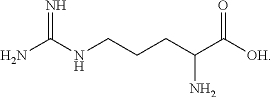 [SMILES: N=C(N)NCCCC(N)C(=O)O]

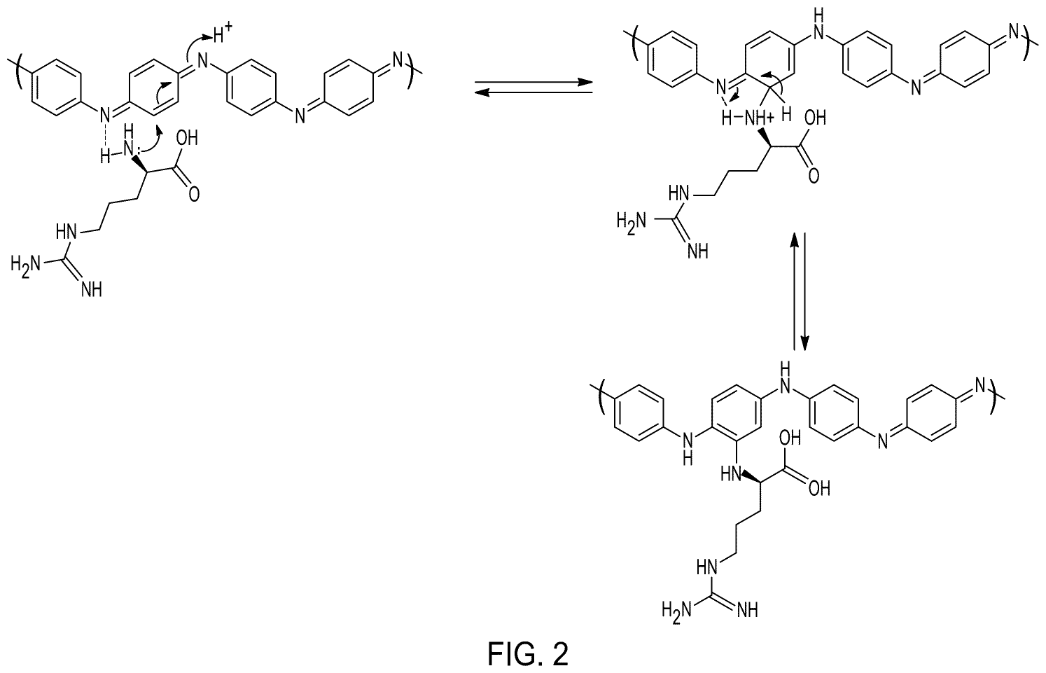

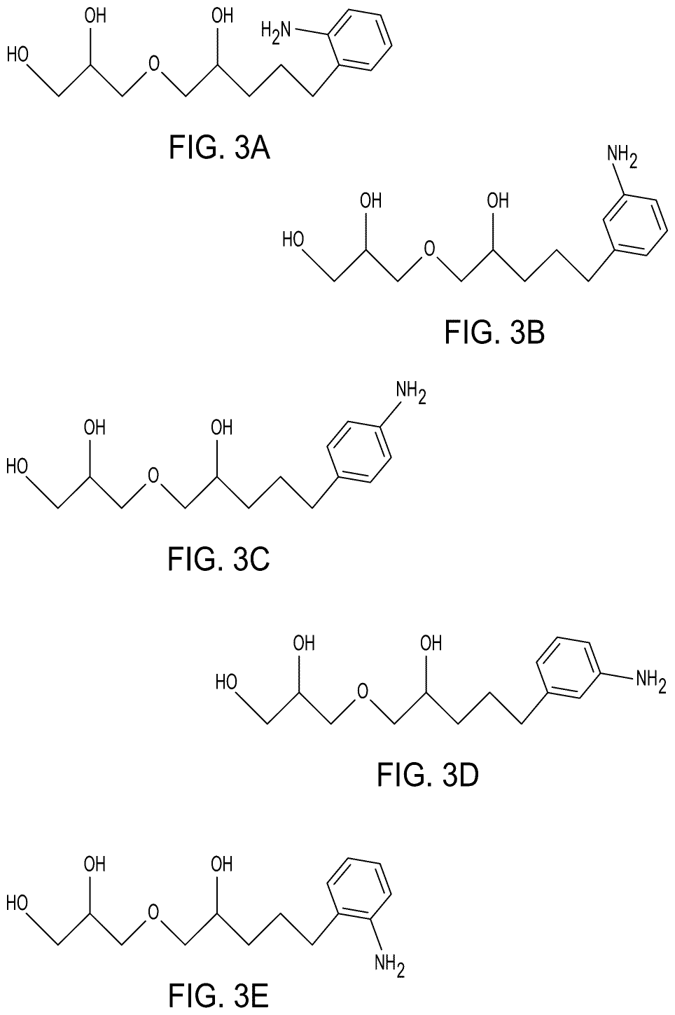

An example of the synthesis of this example of the film 12 is shown in FIG. 2.

Polyaniline is capable of transforming between three different oxidation states (Leucoemeraldine, Emeraldine, Pernigraniline), depending upon whether protonation, oxidation, deprotonation, or reduction is performed. This change in oxidation states can be seen in a voltammogram as redox peaks in positive or negative scan. On applying 1V to polyaniline, the polymer quickly transforms to its fully oxidized state (Pernigraniline). This allows the nucleophilic addition of arginine on the quinoid structure of polyaniline, as shown in FIG. 2.

In another specific example, the opioid 20 to be sensed is morphine; the conductive polymer 16 is polyaniline or a copolymer of polyaniline and polypyrrole; and the anchor molecule is

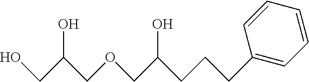 [SMILES: OCC(O)COCC(O)CCCc1ccccc1]

wherein —NH₂ is attached to a carbon of the phenyl ring or in place of one hydrogen of one —CH₂ in the linear chain. The amine group of structure ii) can be attached to the quinoid structure of aniline, in the same manner as shown in FIG. 2.

Each example of the conductive polymer 16 includes a repeating monomer (e.g., aniline), and the anchor molecule 18 is covalently attached to some (but not all) of the repeating monomers in the conductive polymer chain. As such, the repeating monomer and the anchor molecule 18 are present at a predetermined mole ratio within the conductive polymer 16. In an example, the conductive polymer 16 includes the repeating monomer, and the ratio of the repeating monomer to the anchor molecule 18 ranges from 2:1 to 100:1.

In an example, the film 12 has a thickness ranging from about 10 μm to about 500 μm.

The electrode 22 of the opioid sensors 10A, 10B, 10C is positioned in contact with the second of the two opposed surfaces 14B. The electrode 22 that is positioned in contact with the film 12 is the working electrode.

The sensors 10A, 10B, 10C also include a counter electrode 25 and a reference electrode 27 (as shown in FIG. 1B), or a counter/reference electrode 34 (as shown in FIG. 4A). The counter electrode 25 and reference electrode 27 are, or the counter/reference electrode 34 is, physically isolated from the working electrode 22 and from the film 12. As shown in FIG. 1A, the counter and reference electrodes 25, 27 (or the counter/reference electrode 34) of the sensor 10A may be positioned on the substrate 26 a spaced distance from the working electrode 22 and the film 12. Alternatively, the counter and reference electrodes 25, 27 or the counter/reference electrode 34 in the sensor 10A may be positioned on the surface of the substrate 26 that is opposed to the surface that is in contact with the working electrode 22. As shown in FIG. 1D, the counter and reference electrodes 25, 27 are positioned on the substrate 26 such that they are spaced apart from the working electrode 22 and the film 12.

In any of the example sensors 10, each electrode 22, 25, 27, 34 is individually and electrically connected (e.g., via electrical leads and/or wiring) to any suitable device 36 (see FIG. 4A through FIG. 4D) that can measure impedance, which can be used to determine the phase angle. The connections from the electrodes 22, 25, 27, 34 to the measurement device 36 may be direct or through a grip mount convert to a USB port on the measurement device 36. Examples of suitable measurement devices 36 include a potentiostat or an impedance analyzer.

In any of the example sensors 10, each of the working and counter electrodes 22, 25 is independently selected from the group consisting of gold, platinum, palladium, and carbon. As an additional example for the needle sensors 10D, 10E, 10F, 10G, each of the working and counter electrodes 22, 25 may be medical grade stainless steel or platinum-iridium. The reference electrode 27 or counter/reference electrode 34 may be silver/silver chloride.

The thickness of each electrode 22, 25, 27, 34 may range from about 200 nm to about 800 nm. In one example, the thickness of each electrode 22, 25, 27, 34 may range from about 400 nm to about 600 nm.

The size, shape, and configuration of the working electrode 22 will depend, in part, upon the type of sensor and the desired size and shape of the film 12. The size, shape, and configuration of all of the electrode(s) 22, 25, 27, 34 will depend upon the type of sensor 10 and the size and shape of the substrate 26 that supports the electrode(s) 22, 25, 27, 34. In the example shown in FIG. 1A, the working electrode 22 is a branched electrode with multiple arms extending from a main portion (similar to an interdigitated electrode), and the counter and reference electrodes 25, 27 are respectively positioned at opposed ends of the main portion and extend, at least partially, along the outer most branches. In the example shown in FIG. 1B, the working electrode 22 is a single rectangular electrode, the counter electrode 25 is a “J” shaped electrode positioned along one side of the working electrode 22 and whose end hooks around the end of the working electrode 22, and the reference electrode is 27 is a single rectangular electrode positioned along the other side of the working electrode 22.

Some of the sample fluids, e.g., blood and tears, contain electrolytes. In these instances, the fluid adsorbed by the sensor 10A, 10B, 10C may function as the electrolyte between the electrodes 22, 25, 27 (see FIG. 1B). In the in vitro examples (i.e., the sensor is exposed to the sample outside of a user's body), an additional electrolyte, such as phosphate buffered saline, may be added to the sample. In an example, any additional electrolyte may be used that contains similar electrolytes to those inherently contained in the liquid sample or the liquid carrier of the dissolved substance.

The opioid sensors 10A, 10B, 10C may further include a substrate 26. The substrate 26 supports the electrodes 22, 25, 27, 34 and the film 12, either directly or indirectly. Examples of suitable substrates 26 for the sensors 10A, 10B, 10C include polydimethylsiloxane (PDMS), polyethylene terephthalate (PET), polyethylene naphthalate, polyimide, polyene, and poly (methyl methacrylate) (PMMA). Examples of more rigid substrates include glass, silicon, and even paper.

For the opioid sensor 10A (e.g., the patch), the substrate 26 supports the electrodes 22, 25, 27 directly, and supports the film 12 both directly and indirectly. The working electrode 22 is formed on the surface of the substrate 26. The film 12 is formed on the working electrode 22 and on portions of the substrate 26 surface that is exposed between and around the main portion and the branches of the working electrode 22. As such, some of the surface 14B of the film 12 directly contacts the substrate 26 and some of the surface 14B of the film 12 directly contacts the working electrode 22. In this example, the counter and reference electrodes 25, 27 are formed on the same surface of the substrate 26 as the working electrode 22. However, the counter and reference electrodes 25, 27 are physically separated from the film 12 and from the working electrode 22. As such, the film 12 does not contact the counter electrode 25 or the reference electrode 27 (or the counter/reference electrode 34 if used). For the opioid sensor 10A, other portions of the substrate 26, e.g., at the perimeter, may include an adhesive that is suitable for securing the opioid sensor 10A to a person's skin. Any adhesive that is used will not be in contact with the electrodes 22, 25, 27, 34 or the film 12.

Alternatively, the opioid sensor 10A (e.g., the patch), may include the counter electrode 25 and reference electrode 27 or the counter/reference electrode 34 on the surface of the substrate 26 that is opposed to the surface upon which the working electrode 22 and film 12 are formed.

For the opioid sensor 10B (e.g., test strip), the substrate 26 supports the electrodes 22, 25, 27 (or 34 if used), and both the substrate 26 and the electrode 22 support the film 12. In this example, each of the working electrode 22, the counter electrode 25, and the reference electrode 27 (or the counter/reference electrode 34 if used) is positioned on the surface of the substrate 26, and the film 12 is positioned on a portion of the working electrode 22, but not directly on the surface of the substrate 26.

As mentioned, in some examples, the substrate 26 may function as a portion of a housing for the electrodes 22, 24, 25, 27 and the film 12, and a protective coating or lid used in conjunction with the substrate 26 may have an opening (sample input port 28) to receive or adsorb the sample. The protective film may be any non-electrically conductive material that does not dissolve in the sample to which the sensor, e.g., 10B, is exposed. Examples of the protective film include polyethylene, polyvinylchloride, polystyrene, polytetrafluoroethylene, or the like. Examples of the lid 32 include non-electrically conductive polymers or glass, which are inert to the sample. In the example shown in FIG. 1D, the lid 32 may be attached to the substrate 26 using a suitable adhesive.

FIG. 4A through FIG. 4D illustrate different examples of the opioid needle sensors 10D, 10E, 10F, 10G.

In the opioid needle sensor 10D shown in FIG. 4A, the working electrode 22 is a mono-polar needle electrode. The mono-polar needle electrode may be formed of medical grade stainless steel or another medical grade conductive metal.

The mono-polar needle electrode includes a tip 40 and a shaft 42. In this example, the tip 40 and shaft 42 are solid. The tip 40 may be from about 0.5 mm to about 1 mm long and the shaft 42 may be from about 5 cm to about 10 cm long. While several examples have been provided, it is to be understood that other needle dimensions may be used. The tip 40 is a finely sharpened point for insertion into a blood vessel.

The film 12 is positioned on the tip 40 of the mono-polar needle electrode, and an insulating layer 38 is coated on the shaft 42. The insulating layer 38 may be polytetrafluoroethylene (PTFE). The insulating layer 38 may aid in the ease of insertion and removal of the opioid needle sensor 10D into the blood vessel and reducing friction at the insertion point.

The end of the shaft 42 includes an insulated hub 44 that houses the lead wire 46, which attaches the mono-polar needle electrode to a connector (not shown) that electrically connects the lead wire 46 to the device 36 that can measure impedance. In an example, the connector is 5-pin DIN connector.

A separate reference/counter electrode 34 is used with this example sensor 10D. The separate reference/counter electrode 34 may be any of the examples set forth herein and is electrically connected to the device 34.

The opioid needle sensor 10E shown in FIG. 4B is a concentric needle or coaxial needle. In this example, the counter/reference electrode 34 is a cannula of the concentric needle, the working electrode 22 is a wire extending through the cannula, the film 22 is positioned at least on the tip of the wire, and the sensor 10E further includes an insulating material 48 electrically isolating the wire and the cannula.

The counter/reference electrode 34 is a cannula. The cannula may be formed of medical grade stainless steel or another medical grade metal. The size may range from 14 to 26 gauge. The tip of the cannula is a finely sharpened point for insertion into a blood vessel. The counter/reference electrode 34 is electrically attached to the device 36. Alternatively, the cannula of the sensor 10E may be a non-electrically conductive material (e.g., polytetrafluoroethylene (PTFE), an acrylic material, polyethylene, or polypropylene) and a separate counter/reference electrode 34 may be used.

In this example, the working electrode 22 is a wire. The wire may be any of the electrode materials set forth herein, and in one example is a platinum-iridium electrode. The film 12 is positioned at least on the tip of the wire that is exposed at the cannula tip. In the example shown in FIG. 4B, the film 12 coats the entire wire.

The interior of the cannula includes an insulating material 48 that electrically and physically separates the film 12 from the interior surface of the cannula. The insulating material 48 may be polytetrafluoroethylene (PTFE), an acrylic material, polyethylene, epoxy, or polypropylene.

The end of the cannula is attached to the insulated hub 44 that houses the lead wire 46, which attaches the wire working electrode 22 to the connector (not shown) that electrically connects the lead wire 46 to the device 36.

The opioid needle sensor 10F shown in FIG. 4C is similar to the example shown in FIG. 4B, except that the tip of the working electrode 22 includes a barb 50 and the film 12 coats the barb 50. The film 12 may coat the barb 50 without coating the remainder of the wire working electrode 22, or it may coat the entire wire working electrode 22, including the barb 50. In this example, the counter/reference electrode 34 is the cannula and the insulating material 48 electrically isolates the cannula from the wire.

The opioid needle sensor 10G shown in FIG. 4D is bipolar needle electrode, which is another example of a concentric needle. In this example, the reference electrode 27 is a cannula of the concentric needle, the working electrode 22 is a first wire extending through the cannula, the film 22 is positioned at least on the tip of the first wire, the counter electrode 25 is a second wire extending through the cannula, and the sensor 10G further includes the insulating material 48 electrically isolating the first wire, the second wire, and the cannula.

In this example, the reference electrode 27 is the cannula. The cannula may be formed of medical grade stainless steel or another medical grade metal. The size may range from 14 to 26 gauge. The tip of the cannula is a finely sharpened point for insertion into a blood vessel. The reference electrode 27 is electrically attached to the device 36.

In this example, the working electrode 22 is a first wire housed in the cannula. The first wire may be any of the electrode materials set forth herein, and in one example is a platinum-iridium electrode. The film 12 is positioned at least on the tip of the first wire that is exposed at the cannula tip. In the example shown in FIG. 4D, the film 12 coats the entire first wire.

In this example, the counter electrode 25 is a second wire housed in the cannula. The second wire may be any of the electrode materials set forth herein, and in one example is a platinum-iridium electrode.

The first and second wires are positioned at a spaced distance from each other throughout the length of the cannula. The interior of the cannula includes the insulating material 48 that electrically and physically separates the wires from each other and from the cannula. The insulating material 48 may be any of the examples set forth herein.

The end of the cannula is attached to the insulated hub 44 that houses the lead wire 46, which attaches the first wire working electrode 22 to the connector (not shown) that electrically connects the lead wire 46 to the device 36. The second wire also extend through the insulated hub 44 and is electrically connected to the device 36. A separate connection attached the cannula (reference electrode 27 in this example) to the device 36.

A method for making the opioid sensor 10A, 10B, 10C includes printing a working electrode 22 on a substrate 26; and depositing a film 12 over the working electrode 22, the film 12 including a conductive polymer 16 and an anchor molecule 18 attached to the conductive polymer 16, wherein the anchor molecule 18 is to adsorb an opioid 20. The deposition of the film 12 may involve first depositing the conductive polymer 16, and then attaching the anchor molecule 18.

This method may further include printing a counter electrode 25 and a reference electrode 27 or a counter/reference electrode 34 on the substrate 26 a spaced distance from each other and from the working electrode 22. The counter and reference electrodes 25 or the counter/reference electrode 34 may be printed on the same surface of the substrate 26 as the working electrode 22 or on an opposed surface. As described herein, the film 12 may be deposited on the working electrode 22 alone, or on a portion of the substrate 26 such that it is physically separated from the counter electrode 25 and the reference electrode 27 or the counter/reference electrode 34.

The electrode(s) 22, 25, 27, 34 may be printed on the substrate 26 using an electrically conductive ink and a suitable printing process, such as inkjet printing or microcontact printing, or formed using a photolithography process, etc.

The film 12 may be deposited such that it at least overlies the working electrode 22. A deposition process, such as electrochemical deposition, may be used to deposit the conductive polymer 16 on the entire working electrode 22 surface, and then the anchor molecule 18 may be attached thereto.

Electrochemical deposition is particularly desirable for obtaining a uniform layer of the conductive polymer 16. The film 12 may be prepared layer by layer, where the conductive polymer 16 is deposited, and then the anchor molecule 18 is attached. The film 12 may also be deposited so that it coats at least a portion of the substrate 26 surrounding the working electrode 22. If the other electrodes 25, 27 or 34 are/is positioned on the substrate 26, the deposition of the film 12 is controlled so that these electrodes 25, 27 or 34 are not in contact with the film 12.

Anchor molecule attachment takes place via a suitable chemical reaction for the anchor molecule 18 and conductive polymer 16 that are used. In an example, arginine addition to polyaniline or a copolymer of polyaniline and polypyrrole may be performed by applying a suitable voltage to the conductive polymer while it is in the presence of an arginine solution.

A method for making the opioid sensor 10D, 10E, 10F, 10G includes depositing any example of the film 12 disclosed herein at least on a tip of a needle electrode. The needle electrode may be the mono-polar needle electrode or the wire electrodes described herein. The film 12 may be printed on the tip or on the entire needle electrode surface. The needle electrode may alternatively be dipped in the conductive polymer 16. The anchor molecule 18 is then attached as described herein.

For the sensors 10E, 10D, 10E, the film 12 coated working wire electrode, alone or along with the counter electrode 25, is/are then positioned inside the cannula. Any example of the insulating material 48 is introduced into the cannula to prevent electrical shorting. The electronics of the needle sensors 10D-10G are operatively positioned in and through the insulating hub 44.

A method for using any example of the opioid sensor 10 includes exposing a first of two opposed surfaces 14A of a film 12 of the opioid sensor 10 to a fluid suspected of containing an opioid 20, the opioid sensor 10 including: the film 12, which includes the conductive polymer 16 and the anchor molecule 18 attached to the conductive polymer 16 so that it is immobilized at the first of the two opposed surfaces 14A, the anchor molecule 18 to adsorb the opioid 20; a working electrode 22 at least partially in contact with a second of the two opposed surfaces 14B; and a counter electrode 25 or counter/reference electrode 34 electrically connected to, and positioned a spaced distance from, the working electrode 22; and then monitoring the opioid sensor 10 for a change in impedance. During fluid exposure, a frequency of alternative current is applied between the electrodes 22, 25 or 22, 34. In one example, the frequency ranges from about 0.1 Hz to about 100 Hz.

As schematically illustrated in FIG. 1C, the anchor molecule 18 has a strong affinity toward the opioid 20 in the fluid, which increases the delocalization of charge at the interface (between the film 12 and the fluid) with the specific adsorption. The charge delocalization results from two benzene rings in the opioid (e.g., fentanyl or morphine) structure. However, interaction with the anchor molecule 18 further enhances this delocalization with additional positive charge resonating within the amine-amide groups in the anchor molecule 18. This changes the electronic properties of the conductive polymer 16 and further changes the phase angle of the sensor 10.

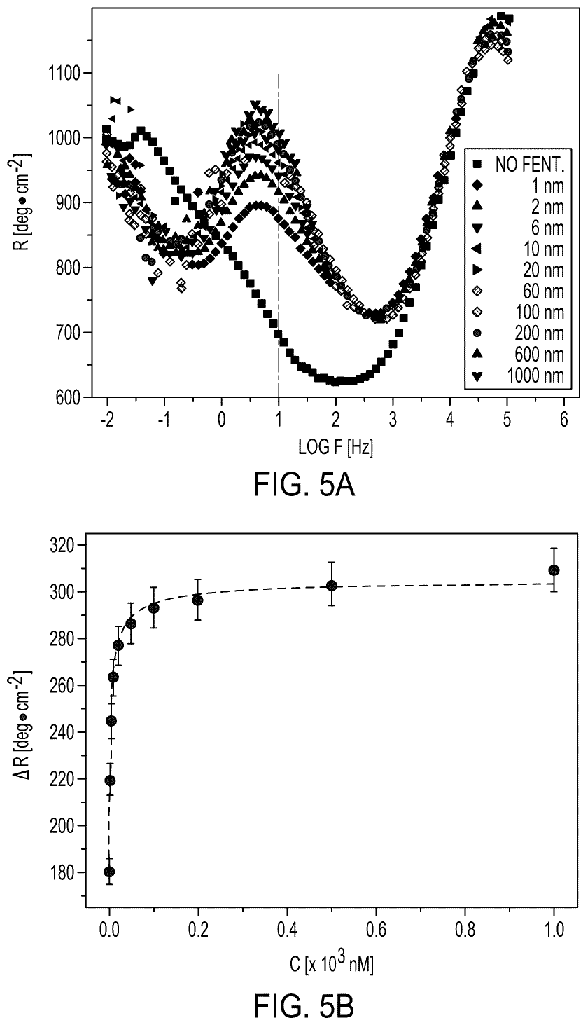

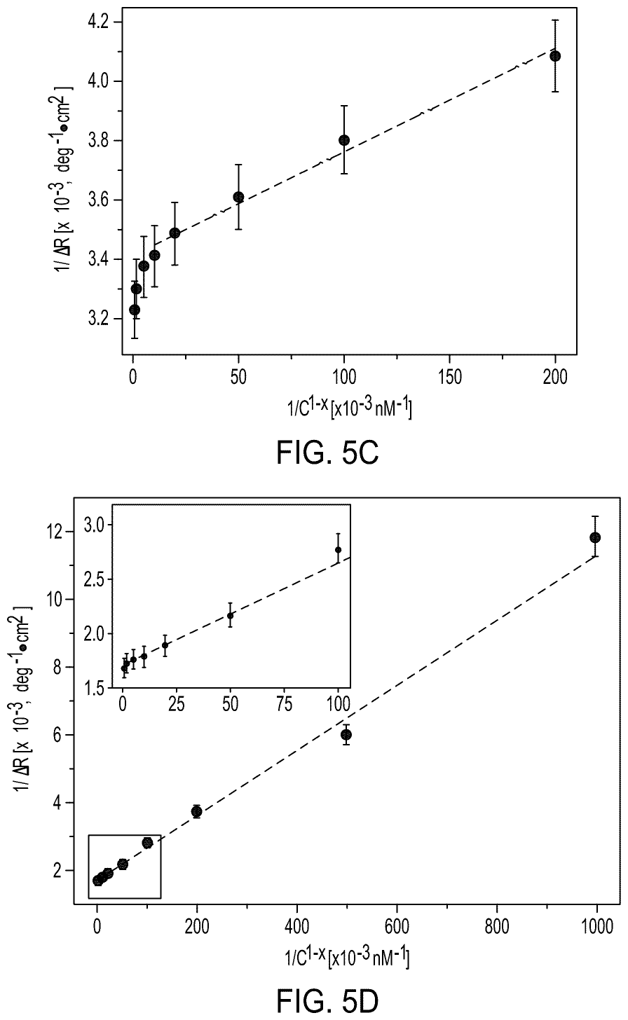

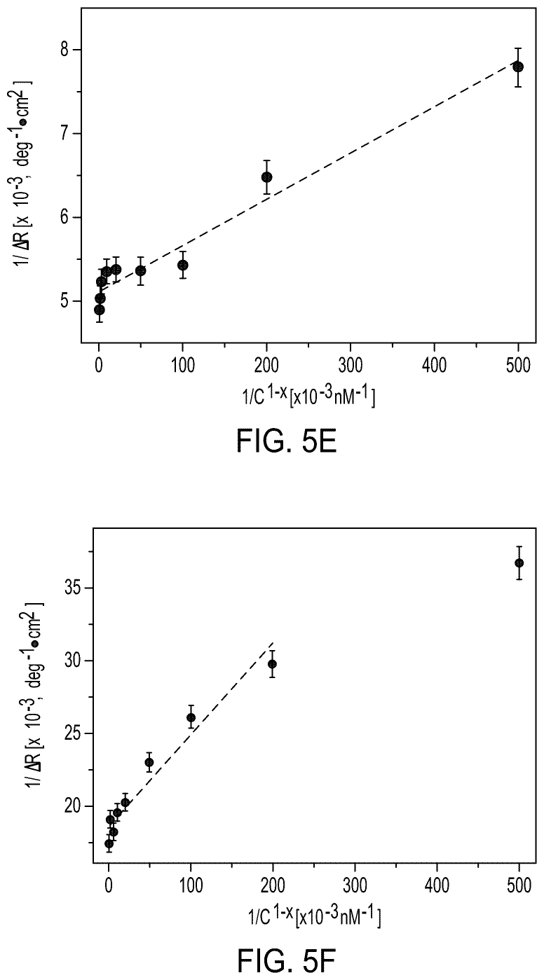

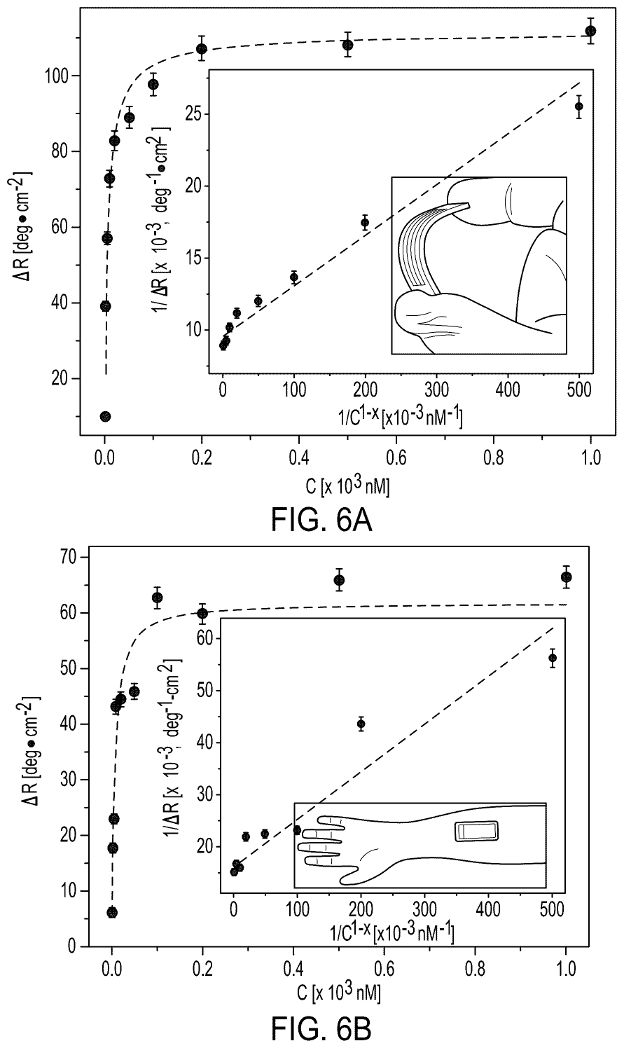

For at least some of the opioids (e.g., fentanyl and morphine), a linear relationship has been observed between the impedance (e.g., the phase angle) and known concentrations of these opioids (see, e.g., FIG. 5C and the inset of FIG. 6B). Thus, the phase angle signal can be measured as a difference between responses (ΔR) when no opioid is present and when a concentration of the opioid is present. Additionally, any unknown opioid concentration can be quantified using the impedance (e.g., phase angle) of the opioid and a linear regression technique (such as Langmuir). The slope of the line from the Langmuir linear regression represents the sensitivity of the phase angle toward the opioid concentration. The linear equation can then be used to determine the opioid concentration in an unknown sample. In particular, the linear fit of the phase angle versus the opioid concentration may be used to determine the unknown opioid concentration because the phase angle signal can be measured using the sensors 10 disclosed herein.

As such, examples of the method include determining a concentration of the opioid 20 based on the change in impedance, and in particular, in phase angle.

In some instances, the output of the opioid sensor 10 is the impedance signal, and the impedance signal may be used, e.g., by a user of the device, to determine the opioid concentration. In this example, the opioid sensor 10 is configured to monitor a change in impedance, and the change in phase angle is determined from the change in impedance.

In other instances, the output of the opioid sensor 10 is the detected opioid concentration. In these instances, the sensor 10 is programmed with the correlation between the impedance (e.g., the phase angle) and the opioid concentration, and is able to output the opioid concentration that correlates with the detected impedance via the device 36.

To further illustrate the present disclosure, examples are given herein. It is to be understood that these examples are provided for illustrative purposes and are not to be construed as limiting the scope of the present disclosure.

### EXAMPLES

### Example 1

Fentanyl was added to different samples of a phosphate buffered saline (PBS) solution in different concentrations ranging from 0 nM to 1000 nM.

An opioid sensor was prepared by printing gold working electrodes on a PDMS substrate in a pattern similar to that shown in FIG. 1A. A polyaniline film was deposited on the electrodes using an electrochemical process. Arginine was attached on polyaniline by dipping the layered structure into an arginine solution and applying 1 V for 10 minutes. A potentiostat was connected to the electrodes for monitoring the phase angle. All potentials were measured using Ag/AgCl as a counter/reference electrode.

The opioid sensor was sequentially exposed to each of the samples. During each sample exposure, the sensor was exposed to a frequency of alternating current (AC) ranging from about 0.01 Hz to about 100,000 Hz, and the phase angle was measured throughout the exposure. The results, in terms of phase angle per electrode surface area (degrees·cm²) per log of frequency (log f [Hz]), are shown in FIG. 5A. As depicted, the response signal is dependent on fentanyl concentration. These results indicate that fentanyl uptake takes place at the interface between the fluid and the film, and that the conductive polymer portion of the film amplifies the signal. The change in the response signal correlated with a change in the amount of fentanyl in the PBS solution, which illustrated the sensor's sensitivity to the amount. It is believed that this is due to the strong affinity of arginine towards fentanyl, which increases the delocalization of charge at the interface with increasing concentration. The charge delocalization results from the two benzene rings in fentanyl structure; and interaction with arginine further enhances this delocalization, with additional positive charge resonating within the amine-amide groups in arginine structure. This changes the electronic properties of polyaniline, which changes the phase angle of the system.

As depicted, the response of the sensor changes with the change in the amount of fentanyl in the (PBS) solution and the change in frequency. FIG. 5B depicts the response of the sensor from FIG. 5A at 10 Hz (i.e., log f=1 Hz) in terms of ΔR, i.e., the difference between the sensor response at no fentanyl (nM) and at each fentanyl concentration.

The physical interaction of fentanyl with the anchor molecules of the film may lead to the formation of a fentanyl monolayer near the interface between the fluid and the sensor. The Langmuir isotherm was used to confirm the mass transfer and physical interaction of fentanyl with the sensor. It was assumed that there were a finite number of identical sites on which the fentanyl monolayer could form. As shown in FIG. 5C, the sensor response followed the Langmuir adsorption relation.

The opioid sensor was also sequentially exposed to human serum (blood), artificial tears, and artificial sweat. The artificial tears were a commercially available solution containing the following active ingredients: polyvinyl alcohol 0.5% and povidone 0.6%. The artificial sweat was prepared by mixing 22 mM urea, 5.5 mM lactic acid, 3 mM NH⁴⁺, 100 mM Na⁺, 10 mM K⁺, 0.4 mM Ca²⁺, 50 μM Mg²⁺, and 25 μM uric acid. Different concentrations of fentanyl were added to these fluids and were exposed to the sensor. The results for the human serum (blood), artificial tears, and artificial sweat are shown, respectively, in FIG. 5D, FIG. 5E, and FIG. 5F. These results illustrate the sensor's sensitivity to fentanyl over a wide concentration range in a variety of fluids.

### Example 2

Two flexible test strip opioid sensors were prepared by printing carbon electrodes on a polytetrafluoroethylene substrate and a PDMS substrate. A polyaniline film was deposited on the carbon electrodes using an electrochemical process. Arginine was attached on polyaniline by dipping the layered into an arginine solution and by applying 1V for 10 minutes. A potentiostat was connected to the electrodes for monitoring the phase angle. All potentials were measured using Ag/AgCl as a counter/reference electrode.

The sensor was bent (see the inset of FIG. 6A) and placed in contact with human serum (blood), and was also placed on an artificial arm soaked in the artificial sweat (see the inset of FIG. 6B). The phase angles were recording and the change in phase angle was calculated (taking into account the electrode surface area) and plotted against the known concentration of fentanyl in the samples. The results for the human serum are shown in FIG. 6A and the results for the artificial sweat are shown in FIG. 6B. The results are similar to those shown in FIG. 5D and FIG. 5F, respectively. These results indicate that interferences, e.g., due to sensor bending, were negligible.

### Example 3

An opioid sensing needle electrode was prepared by depositing a polyaniline film on a stainless steel or carbon needle electrode using an electrochemical process. Arginine was attached on polyaniline by dipping the coated needle into an arginine solution and applying 1 V for 10 minutes. A potentiostat was connected to the electrodes for monitoring the phase angle. All potentials were measured using Ag/AgCl as a counter/reference electrode.

Fentanyl was added to different samples of a phosphate buffered saline (PBS) solution in different concentrations ranging from 0 μM to 0.1 μM.

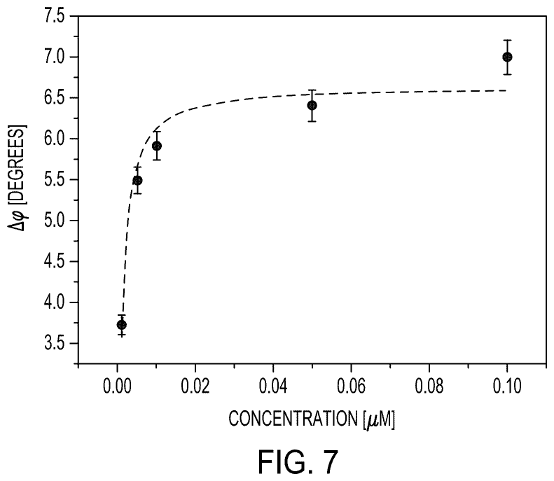

The opioid sensing needle electrode was sequentially exposed to each of the samples. During each sample exposure, the opioid sensing needle electrode was exposed to a frequency of alternating current (AC) ranging from about 0.01 Hz to about 100,000 Hz, and the phase angle was measured throughout the exposure. The results, in terms of phase angle (degrees) versus the concentration ([μM]), are shown in FIG. 7. As depicted, the response signal is dependent on fentanyl concentration. These results indicate that fentanyl uptake takes place at the interface between the fluid and the film, and that the conductive polymer portion of the film amplifies the signal. The change in the response signal correlated with a change in the amount of fentanyl in the PBS solution, which illustrated the sensor's sensitivity to the amount. It is believed that this is due to the strong affinity of arginine towards fentanyl, as described herein. This changes the electronic properties of polyaniline, which changes the phase angle of the system.

As illustrated in all of the examples, the opioid sensors 10 disclosed herein exhibit high specificity to a particular opioid. Thus, the sensors 10 disclosed herein also enable the highly sensitive detection and quantification of the concentration of an opioid (e.g., fentanyl, morphine) in bodily fluids or liquid samples of a substance. The opioid sensors 10 also exhibit resistant to interference.

Moreover, the opioid sensors 10 disclosed herein also enable the rapid detection and quantification of the concentration of the opioid, with response times ranging from 1 minute to about 10 minutes.

It is to be further understood that the ranges provided herein include the stated range and any value or sub-range within the stated range, as if such value(s) or sub-range(s) were explicitly recited. For example, a range from about 10 μm to about 500 μm should be interpreted to include not only the explicitly recited limits of from about 10 μm to about 500 μm, but also to include individual values, such as 15.5 μm, 100 μm, 380 μm, 475.75 μm, etc., and sub-ranges, such as from about 20 μm to about 480 μm, from about 10 μm to about 250 μm, etc. Furthermore, when “about” is utilized to describe a value, this is meant to encompass minor variations (up to +/−10%) from the stated value.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

In describing and claiming the examples disclosed herein, the singular forms “a”, “an”, and “the” include plural referents unless the context clearly dictates otherwise.

While several examples have been described in detail, it is to be understood that the disclosed examples may be modified. Therefore, the foregoing description is to be considered non-limiting.

What is claimed is:

1. An opioid sensor, comprising:
a film having two opposed surfaces, the film including:
a conductive polymer; and
an anchor molecule attached to the conductive polymer so that it is immobilized at a first of the two opposed surfaces, the anchor molecule to adsorb an opioid;
a working electrode at least partially in contact with a second of the two opposed surfaces; and
a counter electrode or counter/reference electrode electrically connected to, and positioned a spaced distance from, the working electrode.

2. The opioid sensor as defined in claim 1, wherein the conductive polymer is selected from the group consisting of polyaniline, and a copolymer of polyaniline and polypyrrole.

3. The opioid sensor as defined in claim 1, wherein the anchor molecule is selected from the group consisting of:
i) 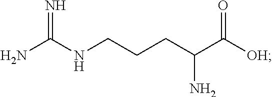 [SMILES: N=C(N)NCCCC(N)C(=O)O]
and
ii) 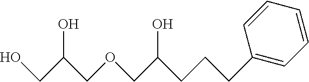 [SMILES: OCC(O)COCC(O)CCCc1ccccc1]
wherein —NH₂ is attached to a carbon of the phenyl ring or in place of one hydrogen of one —CH₂ in the linear chain.

4. The opioid sensor as defined in claim 1, wherein:
the opioid is fentanyl;
the conductive polymer is polyaniline; and
the anchor molecule is

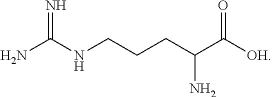 [SMILES: N=C(N)NCCCC(N)C(=O)O]

5. The opioid sensor as defined in claim 1, wherein:
the opioid is morphine;
the conductive polymer is polyaniline or a copolymer of polyaniline and polypyrrole; and
the anchor molecule is 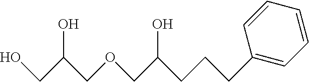 [SMILES: OCC(O)COCC(O)CCCc1ccccc1]
wherein —NH₂ is attached to a carbon of the phenyl ring or in place of one hydrogen of one —CH₂ in the linear chain.

6. The opioid sensor as defined in claim 1, wherein each of the working electrode and the counter electrode or counter/reference electrode is independently selected from the group consisting of gold, platinum, palladium, and carbon.

7. The opioid sensor as defined in claim 1, wherein:
the conductive polymer includes a repeating monomer; and
a ratio of the repeating monomer to the anchor molecule ranges from 2:1 to 100:1.

8. The opioid sensor as defined in claim 1, further comprising a substrate, wherein:
the working electrode is positioned on the substrate;
the film is positioned on the working electrode; and
the counter/reference electrode is positioned on the substrate a spaced distance from the working electrode.

9. The opioid sensor as defined in claim 1, further comprising:
a substrate; and
a reference electrode, wherein:
the working electrode is positioned on the substrate;
the film is positioned on the working electrode;
the counter electrode is positioned on the substrate a spaced distance from the working electrode; and
the reference electrode is positioned on the substrate a spaced distance from each of the working electrode and the counter electrode.

10. The opioid sensor as defined in claim 1, wherein:
the working electrode is a mono-polar needle electrode; and
the film is positioned on a tip of the mono-polar needle electrode.

11. The opioid sensor as defined in claim 10, further comprising an insulating layer coating a shaft of the mono-polar needle electrode.

12. The opioid sensor as defined in claim 1, wherein:
the opioid sensor is a concentric needle;
the counter/reference electrode is a cannula of the concentric needle;
the working electrode is a wire extending through the cannula;
the film is positioned at least on a tip of the wire; and
the opioid sensor further comprises an insulating material electrically isolating the wire and the cannula.

13. The opioid sensor as defined in claim 12, wherein:
the tip of the wire includes a barb; and
the film coats the barb.

14. The opioid sensor as defined in claim 1, wherein:
the opioid sensor is a concentric needle;
a cannula of the concentric needle is a reference electrode;
the working electrode is a first wire extending through the cannula;
the counter electrode is a second wire extending through the cannula;
the film is positioned at least on a tip of the first wire; and
the opioid sensor further comprises an insulating material electrically isolating the first wire, the second wire, and the cannula.

15. The opioid sensor as defined in claim 1, further comprising a potentiostat or an impedance measurement device electrically connected to the working electrode and the counter electrode or counter/reference electrode.

16. The opioid sensor as defined in claim 1, wherein the film has a thickness ranging from about 10 μm to about 500 μm.

17. The opioid sensor as defined in claim 1, wherein the conductive polymer is selected from the group consisting of poly (2,3-dimethylaniline), polyethoxyaniline, poly (o-anisidine), poly (3,4-ethylenedioxythiophene), polyacetylene, polypyrrole, polythiophene, poly (para-phenylene), poly (phenylenevinylene), polyfuran, and copolymers thereof.

18. A method, comprising:
exposing a first of two opposed surfaces of a film of an opioid sensor to a fluid suspected of containing an opioid, the opioid sensor including:
the film including:
a conductive polymer; and
an anchor molecule attached to the conductive polymer so that it is immobilized at the first of the two opposed surfaces, the anchor molecule to adsorb the opioid; and
a working electrode at least partially in contact with a second of the two opposed surfaces; and
a counter electrode or counter/reference electrode electrically connected to, and positioned a spaced distance from, the working electrode; and then
monitoring the opioid sensor for a change in impedance.

19. The method as defined in claim 18, further comprising determining a change in phase angle from the change in impedance.

20. The method as defined in claim 19, further comprising determining a concentration of the opioid based on the change in phase angle.

21. The method as defined in claim 20, further comprising quantifying the concentration of the specific gas molecule based on the change in the phase angle.

22. A method, comprising:
printing a working electrode on a substrate; and
depositing a film on the working electrode, the film including:
a conductive polymer; and
an anchor molecule attached to the conductive polymer, wherein the anchor molecule is to adsorb an opioid.

23. The method as defined in claim 22, further comprising printing a counter electrode and a reference electrode or a counter/reference electrode on the substrate a spaced distance from each other and from the working electrode.

24. The method as defined in claim 22, wherein the film is deposited on a portion of the substrate, but is physically separated from the counter electrode and the reference electrode or the counter/reference electrode.

25. A method comprising:
depositing a film at least on a tip of a needle electrode, the film including:
a conductive polymer; and
an anchor molecule attached to the conductive polymer, wherein the anchor molecule is to adsorb an opioid.
